(19) United States
(12) Patent Application Publication
(10) Pub. No.: US 2006/0142411 A1
(43) Pub. Date: Jun. 29, 2006

(54) Instant tooth whitening with silicone resin and silicone adhesive

(76) Inventors: Sayed Ibrahim, Somerset, NJ (US); Suryakant Patel, Bridgewater, NJ (US); Suman K. Chopra, Dayton, NJ (US); Michael Prencipe, West Windsor, NJ (US)

Correspondence Address: COLGATE-PALMOLIVE COMPANY, 909 RIVER ROAD, PISCATAWAY, NJ, 08855, US

(21) Appl. No.: 11/025,583

(22) Filed: Dec. 29, 2004

(51) Int. Cl. A61K 6/10; C08L 83/04; B32B 27/06

(52) U.S. Cl. 523/109; 524/588

(57) ABSTRACT

A tooth whitening composition for imparting a natural white appearance to dental enamel, comprising: a whitening particulate comprising hydroxyapatite and a delivery system comprising a silicone resin and a silicone adhesive. Methods of imparting whiteness to a tooth in a mammal are also provided.

### INTRODUCTION

The present invention relates to oral care compositions and methods. In various embodiments, this application relates to tooth whitening compositions comprising whitening particulates and a silicone based delivery system.

Many individuals desire a “bright” smile and white teeth, and consider dull and stained teeth cosmetically unattractive. Unfortunately, without preventive or remedial measures, stained teeth are almost inevitable due to the absorbent nature of dental material. Everyday activities such as smoking or other oral use of tobacco products, and eating, chewing, or drinking certain foods and beverages (in particular coffee, tea, and red wine) cause undesirable staining of surfaces of teeth. Staining can also result from microbial activity, including that associated with dental plaque. The chromogens or color causing substances in these materials become part of the pellicle layer and can permeate the enamel layer. Even with regular brushing and flossing, years of chromogen accumulation can impart noticeable tooth discoloration.

Dental gel compositions containing active oxygen liberating ingredients such as hydrogen peroxide, urea peroxide, percarbonates, and perborates of alkali and alkaline earth metals have been disclosed in the prior art for whitening teeth. Such gel compositions although effective for whitening teeth require multiple applications of the gel over a period of one to two weeks to provide the desired whitening effect and in some instances requires a skilled professional to apply the gel. However, in many situations it is desirable to cosmetically improve the appearance of the teeth, that is whiten the teeth rapidly and temporarily as in immediate anticipation of an important public appearance or social event.

There is a continuing need for a rapid temporary tooth whitening system which can be safely applied by the wearer and without the need for specially trained dental personnel. The tooth whitener should be easily applied to the teeth and once applied to the teeth, be able to resist removal by mastication or solvating by foods and beverages for a period for at least 5 to 8 hours, but should be able to be easily removed by a wearer without harming the tooth. In this respect the prior art discloses tooth whitening coatings which can be applied to the teeth as a beauty aid providing immediate temporary whitening when applied to dental enamel in the same manner as one would apply a nail polish to nails. However, there continues to be a need for improved temporary whitening compositions that are effective, durable, and impart a “natural” look to the user's teeth.

### SUMMARY

In various embodiments, the present invention provides a tooth whitening composition for imparting a natural white appearance to dental enamel, comprising a whitening particulate comprising hydroxyapatite; and a delivery system comprising a silicone resin and a silicone adhesive.

In other embodiments, the present invention also provides methods for imparting whiteness to a tooth in a mammal, comprising applying to the tooth a composition, comprising a whitening particulate comprising hydroxyapatite; and a delivery system that comprises a silicone resin and a silicone adhesive.

The present invention further provides tooth whitening compositions, comprising a whitening particulate comprising hydroxyapatite; and a delivery system comprising the silicone resin trimethylsiloxysilicate and at least one silicone adhesive wherein the ratio of trimethylsiloxysilicate to adhesive provides a strong and flexible layer when the composition is contacted with a tooth surface, wherein after application to a tooth the composition forms a strong, flexible, and hard layer over a tooth surface.

It has been discovered that compositions and methods of this invention afford advantages over temporary whitening compositions among those known in the art including one or more of: enhanced whitening efficacy and aesthetics, and enhanced stability in the oral cavity including, improved adherence of the whitening composition to the tooth surface in the presence of saliva. Further uses, benefits and embodiments of the present invention are apparent from the description set forth herein.

### DESCRIPTION

The following description of the preferred embodiment(s) is merely exemplary in nature and is in no way intended to limit the invention, its application, or uses.

The following definitions and non-limiting guidelines must be considered in reviewing the description of this invention set forth herein. The headings (such as “Introduction” and “Summary”) and sub-headings (such as “Compositions” and “Methods”) used herein are intended only for general organization of topics within the disclosure of the invention, and are not intended to limit the disclosure of the invention or any aspect thereof. In particular, subject matter disclosed in the “Introduction” may include aspects of technology within the scope of the invention and may not constitute a recitation of prior art. Subject matter disclosed in the “Summary” is not an exhaustive or complete disclosure of the entire scope of the invention or any embodiments thereof. Classification or discussion of a material within a section of this specification as having a particular utility (e.g., as being a “system” or “carrier”) is made for convenience, and no inference should be drawn that the material must necessarily or solely function in accordance with its classification herein when it is used in any given composition.

The citation of references herein does not constitute an admission that those references are prior art or have any relevance to the patentability of the invention disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features or other embodiments incorporating different combinations the stated of features. Specific Examples are provided for illustrative purposes of how to make and use the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word “include,” and its variants, is intended to be non-limiting, such that recitation of items in a list is not to the exclusion of other like items that may also be useful in the materials, compositions, devices, and methods of this invention.

As referred to herein, all compositional percentages are by weight of the total composition, unless otherwise specified.

“A” and “an” as used herein indicate “at least one” of the item is present. As used herein, the term “about,” when applied to the value for a parameter of a composition or method of this invention, indicates that the calculation or the measurement of the value allows some slight imprecision without having a substantial effect on the chemical or physical attributes of the composition or method. If, for some reason, the imprecision provided by “about” is not otherwise understood in the art with this ordinary meaning, then “about” as used herein indicates a possible variation of up to 5% in the value.

Tooth Whitening Compositions

In accordance with various embodiments of the present invention, a whitening composition is provided for imparting a natural white appearance to a tooth surface. The tooth surface is generally comprised of dental enamel, and the whitening composition contacts and preferably adheres to the dental enamel to impart an immediate discernable whitening effect, thus rapidly altering the color of the surface of the tooth. In certain embodiments, the present invention provides methods for whitening a tooth surface by using compositions of the present invention. As referred to herein, “tooth” or “teeth” refers to natural teeth, dentures, dental plates, fillings, caps, crowns, bridges, dental implants, and the like, and any other hard surfaced dental prosthesis either permanently or temporarily fixed within the oral cavity. As used herein, “whitening” refers to a change in visual appearance of a tooth, preferably such that the tooth has a brighter shade. Increase in whiteness of a dental surface can be observed visually, for example with the aid of color comparison charts or gauges, or measured by colorimetry, using any suitable instrument such as a Minolta Chromameter, e.g., model CR-400 (Minolta Corp., Ramsey, N.J.). The instrument can be programmed, for example, to measure Hunter Lab values or L*a*b* values according to the standard established by the International Committee of Illumination (CIE). The L*a*b* system provides a numerical representation of three-dimensional color space where L* represents a lightness axis, a* represents a red-green axis and b* represents a yellow-blue axis. The L* and b* axes are typically of greatest applicability to measurement of tooth whiteness. Increase in whiteness can be computed from differences in L*, a* and b* values before and after treatment, or between untreated and treated surfaces. A useful parameter is ΔE*, calculated as the square root of the sum of the squares of differences in L*, a* and b* values, using the formula:
ΔE*=[(ΔL*)²+(Δa*)²+(Δb*)²]^{1/2}
A higher value of ΔE* indicates greater increase in whiteness. In various embodiments, the method of the present invention can effect a ΔE* of at least about 1, or at least about 3, or at least about 4, or at least about 5.

In one embodiment of the present invention, the whitening composition comprises a whitening particulate, comprising hydroxyapatite and a delivery system comprising a silicone resin and a silicone adhesive. The whitening composition may comprise additional ingredients, as recognized by one of skill in the art. The whitening composition may be incorporated into a dental strip or film form, or into a liquid product which may be applied to the surface of a tooth by manual application. In another embodiment, methods of whitening a surface of a tooth are provided.

Delivery System

The delivery system is operable to disperse and adhere the whitening particulates to a tooth surface to provide a natural white appearance. The delivery system comprises a combination of a silicone resin and a silicone adhesive, which thus blends a strong and rigid film-forming resin with a soft elastomeric matrix adhesive. In preferred embodiments, the combination of the strong resins and soft adhesives provides an improved flexible film that is able to withstand conditions of the oral environment (temperature, moisture, pH), as well as physical agitation for an extended period of time. In various embodiments, the combination or ratios of the relative amounts of silicone resin and silicone adhesive in the delivery system may be adjusted to provide one or more of: 1) a desired physical and chemical stability of the composition; 2) a desired level of mucoadhesiveness related to the tackiness of the composition; 3) a desired level of flexibility of the composition; 4) a desired level of hardness of the composition; and 5) control over conditions inducing disintegration of the compositions as applied to a tooth surface which further relates to a dissolution rate. For example, an increased level of silicone resin as compared to the adhesive in the delivery system will increase the hardness and rigidity of the composition. An increase in the level of silicone adhesive as compared to the resin will increase the tackiness level of the composition. Modifications in either component may influence the removal technique employed (e.g. brushing, alcohol rinse, peel off, etc.).

One skilled in the art appreciates that the selection of the specific type of silicone resins and silicone adhesives will also impact the composition characteristics.

Resins

A “siloxane polymer” as used herein, generally refers to a cross-linked polymer that has a basic backbone of silicon and oxygen with side constituent groups that may be the same or different, generally described by the structural repeating unit (—O—SiRR′—)ₙ, where R and R′ may be the same or different side constituent groups, and n may be any value above 2 designating the repetition of the structural repeating unit (SRU) in the polymer backbone. Siloxane polymers are also known in the art as “silicone” polymers. Siloxane polymers may include polyheterosiloxanes, where side groups and/or structural repeating units may be different entities (having different side constituent groups), such as, for example, the siloxane co-polymer described by the nominal SRU formula (—O—SiRR′)ₙ—(—O—Si—R″R′″)ₘ, wherein R and R′ are distinct side groups from R″ and R′″. Further R and R′ may be different from one another, likewise the same may be true for R″ and R′″. Such siloxane polymers may terminate in any variety of terminal groups, such as for example, trimethyl silyl ((CH₃)₃Si) terminated siloxane.

Silicone resins as referred to herein are highly crosslinked polymeric siloxane systems. The crosslinking is introduced through the incorporation of tri-functional and tetra-functional silanes with mono-functional or di-functional (or both) silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units (and hence a sufficient level of crosslinking) such that they dry down to a rigid or hard film are considered to be silicone resins. The ratio of oxygen atoms to silicon atoms is indicative of the level of crosslinking in a particular silicon material. Silicone resins generally have at least about 1:1 oxygen atom per silicon atom. Typical silanes used in the manufacture of silicone resins are the monomethyl, dimethyl, monophenyl, diphenyl, methylphenyl, monovinyl, and methylvinyl-chlorosilanes and tetrachlorosilanes. Preferred resins are the methyl, ethyl or phenyl substituted silicone resins.

Silicone resins can be identified according to the “MDTQ” nomenclature where the silicone resin is described according to the presence of various siloxane monomer units. General trends in the MDTQ system also provide insight into the properties of the silicone resin. The symbol M denotes the mono-functional unit ((CH₃)₃SiO)_{0.5} having only one available oxygen for binding. The M units are generally used for end blocking or terminal groups. D denotes the di-functional unit (CH₃)₂SiO having two available oxygen groups. The D units are generally used as chain extenders. The T denotes the tri-functional unit ((CH)₃SiO)_{1.5} having three available oxygens for binding. The T units are generally used to design silicone resins which are cage shaped or three dimensional. Q denotes the quadra- or tetra-functional unit SiO₂ having four available oxygen groups. The MDTQ standard substituent methyl may be substituted for other groups including vinyl, phenyl, amino, hydroxyl, etc. The differences in substituent groups are denoted using primes (e.g., M′). The inclusion of these alternate substituent groups may increase the resin compatibility with other organic materials. The description of the silicone resin is complete with the notation of any non-methyl substituent groups and molar ratios of the various units, either in terms of subscripts to the symbols indicating the total number of each type of unit in the silicone, or an average thereof, or as specifically indicated ratios in combination with molecular weight.

In certain embodiments, preferred silicone resins are MQ resins, such as the MQ resins disclosed in U.S. Pat. No. 6,423,322, to Fry, issued Jul. 23, 2002 and U.S. Pat. No. 5,330,747, to Krysik, issued Jul. 19, 1994. The MQ resins are known for having film forming attributes, fixative properties, strength enhancing properties, and hydrophobizing properties. Such MQ resins are the co-hydrolysis products of tetraalkoxy-silane and trimethylethoxy-silane and the resins are referred to as trimethylsiloxysilicate. As with any silicone resin, hydroxy and alkoxy functional groups may be present in the resin structure as a result of processing. The average molecular weight of the MQ resin may be adjusted via the ratio of M to Q units. The trimethylsiloxysilicate may be a three dimensional network of polysilicic acid units, which are endblocked with trimethylsilyl groups. Examples of suitable silicone resins are commercially available under the tradenames SR 1000, SS 4230, and SS4267 from the General Electric Company of Waterford, N.Y., U.S.A. and Wacker TMS 803 and 804 available from Wacker Silicones Corporation of Adrian, Mich., U.S.A. As stated above, it is understood that the silicone resin may comprise other endblocking or terminal groups, such as amine or hydroxyl groups.

In various embodiments, silicone resins that are useful herein are soluble in organic solvents such as alcohols (e.g., methanol), esters such as ethyl or propyl acetate, mineral oils, waxes, isoparaffins, aromatic solvents such as toluene, and silicone fluids including dimethicone and cyclosiloxanes, and mixtures thereof.

In various embodiments, the silicone resin is present in the composition at from about 0.1% to about 50% by weight, preferably from about 5% to about 20% by weight.

Adhesives

In various embodiments of the present invention, the whitening composition comprises an adhesive in the delivery system, which serves multiple functions, including enhancing adherence of the whitening composition to the surface of the tooth to be whitened. The adhesives are optimized for adhering to the teeth, resisting adherence to non-tooth oral surfaces, and remaining attached to the teeth for an extended time (e.g. 5 to 8 hours or 3 to 7 days). Optimization of such aspects may be achieved from varying the physical and chemical properties of a single adhesive or combining different adhesives as discussed later herein.

In such embodiments, the adhesive generally comprises one or more organic polymers or copolymers that are dispersed or dissolved in a volatile solvent. A variety of organic polymers are useful as adhesives with the present invention and one preferred class of adhesive polymers comprises siloxane polymers. Many such silicone polymers are commercially available. In various embodiments, a preferred silicone-based adhesive polymer is a polyorganosiloxane. One such polyorganosiloxane is produced by condensing a silicone resin and an organosiloxane such as a polydiorganosiloxane. In certain embodiments of the present invention, the adhesive polymers in the composition are those in which a whitening particulate can be dispersed and are well known in the art. Such polymers are an elastomeric, tacky material, adhesion of which to dental enamel surfaces can be varied by altering the ratio of silicone resin to polydiorganosiloxane in the copolymer molecule, and/or by altering the terminal group (e.g. substituting a more sticky or tacky hydroxyl terminal group with a less tacky amine terminal group). Preferably, the polymers are pressure sensitive polymers specifically designed for pharmaceutical use and are permeable to many drug compounds. In one such embodiment, the silicone polymers are the copolymer product of mixing a silanol terminated polydiorganosiloxane such as polydimethyl siloxane with a silanol-containing silicone resin whereby the silanol groups of the polydiorganosiloxane undergo a condensation reaction with the silanol groups of the silicone resin so that the polydiorganosiloxane is lightly cross-linked by the silicone resin (that is, the polydiorganosiloxane chains are bonded together through the resin molecules to give chain branching and entanglement and/or a small amount of network character) to form the silicone polymers. The silanol terminated groups may be substituted with amine groups, for example, to make the siloxane soluble in amine compatible solvents and/or to reduce the tackiness of the adhesive polymer. The silanol may also be substituted with terminal hydroxyl groups to increase adhesiveness of the adhesive polymer.

To promote a cross-linking reaction, a catalyst can be added, for example, an alkaline material, such as ammonia, ammonium hydroxide or ammonium carbonate, can be mixed with the silanol-terminated polydiorganosiloxane and the silicone resin. Copolymerization of the silicone resin with the silanol terminated polydiorganosiloxane, results in a polymer composition having self-adhering properties and cohesive properties typical of a soft elastomer matrix like a pressure sensitive polymer. Such copolymerization lends improved properties over the typical hard, non-elastomeric properties of other silicone resins. In one embodiment, adhesive polymers used in the whitening composition are available from the Dow-Corning Company (Midland, Mich., USA) under the brand name BIO-PSA. BIO-PSA polymers may include silanol, hydroxyl, and amine terminal groups.

The modification of a ratio of silicone resin to polydiorganosiloxane modifies the tackiness of the siloxane adhesive polymer. In various embodiments, this ratio can be in the range of about 70:30 to about 50:50. For example, the BIO-PSA silicone sold by Dow-Corning is available in three silicone resin to silicone polymer ratios namely, 65/35 (low tack), 60/40 (medium tack), 55/45 (high tack). The rapid tooth whitening composition in some embodiments according to the invention contains an organic adhesive dispersed or dissolved in a volatile solvent. For example, a polyorganosiloxane pressure sensitive adhesive can be dissolved in either alkyl acetate (e.g. propyl or ethyl acetate), dimethicone, and/or amine compatible solvents.

In addition to the aforementioned silicone resin and silicone adhesives, additional adhesive polymers can include those such as Carbomers, (carboxymethylene polymers including acrylic acid polymers and acrylic acid copolymers). Carboxypolymethylene is a slightly acidic vinyl polymer with active carboxyl groups. A carboxypolymethylene preferred for use in the practice of the present invention is a copolymer of acrylic acid cross linked with approximately 0.75% to approximately 1.5% polyallyl sucrose that is sold under the trade designation Carbopol 934, 974 by B.F. Goodrich. The additional adhesive is preferably a natural resin, particularly a resin selected from shellac resins, colophonium resins and modified colophonium resins. Another suitable group of adhesives is organic polymeric compositions represented by the group of alkyl resins, polyvinyl acetaldehydes, polyvinyl alcohols, polyvinyl acetates, poly(ethylene oxide), polyacrylates, ketone resins, polyvinylpyrrolidone, polyvinylpyrrolidone/vinyl acetate copolymer, polyethylene glycols of 200 to 1000 molecular weight and polyoxyethylene/polyoxopropylene block copolymers (Polyox), and silicone resins. As appreciated by one of skill in the art, the adhesive polymers may comprise copolymers or mixtures of polymers, including the adhesive polymers described herein.

As appreciated by one skilled in the art, a combination of silicone adhesives may be used to achieve the desired level of tack (e.g., a high tack and a medium tack combination). The combination may also include silicone adhesives having different carriers or functional groups, such as a combination of an amine compatible silicone adhesive and a silicone adhesive in a dimethicone solvent. The combination of adhesives in the delivery system provides sufficient adhesion of the whitening particles to the dental surfaces and promotes flexibility and proper fit of the composition against the teeth.

In various embodiments, an adhesive polymer is present in the liquid whitening compositions of the present invention at a concentration of about 0.5% to about 80% by weight and preferably about 20% to about 50% by weight. In certain embodiments, an adhesive polymer comprises: a pressure sensitive adhesive is present at about 10% to about 30% by weight, preferably about 20% by weight; and additionally either of: 1) a medium tack adhesive is present at about 10% to about 30% by weight or 2) a low tack silicone adhesive at about 10% to about 20% by weight, preferably at 20% by weight.

Adhesion Enhancing Agents

Adhesion enhancing agents are used in the compositions of the present invention to enhance the adhesive properties of the delivery system and include inorganic materials as well as organic natural and synthetic polymers. It is understood that while general attributes of each of the above categories of polymers and whitening composition ingredients may differ, there may some common attributes and any given material may serve multiple purposes within two or more of such categories of ingredients. As such, in certain embodiments, adhesion enhancing agents may be incorporated into the whitening composition nominally as part of the adhesive polymer, however may likewise be classified as an adhesion enhancing agent.

Inorganic adhesion enhancing materials include amorphous silica compounds which function as thickening agents include colloidal silica compounds available under trademarks such as Cab-o-sil fumed silica manufactured by Cabot Corporation and distributed by Lenape Chemical, Brook, N.J., USA; Zeodent 165 from J.M. Huber Chemicals Division, Havre de Grace, Md., USA; and Sylox 15 also known as Sylodent 15, available from Davison Chemical Division of W.R. Grace Corporation, Baltimore, Md., USA.

A preferred amorphous, fumed silica compound is obtainable in powder form from the Degussa Company (Hopewell, Va., USA) under the trade designation “Aerosil”. Various types of Aerosils are available depending on the variations in the manufacturing process. Aerosil 200 is a hydrophilic fumed silica, having a surface area of about 200 square meters per gram, an average particle size of about 10-12 nanometers and a compacted apparent density of about 50 g/l and is preferred for use in the practice of the present invention.

Organic polymers useful as adhesion enhancing agents useful in the practice of the present invention include hydrophilic polymers such as Carbomers such as carboxymethylene polymers such as acrylic acid polymers, and acrylic acid copolymers, as previously described in the context of adhesive polymers. Carboxypolymethylene is a slightly acidic vinyl polymer with active carboxyl groups. A carboxypolymethylene preferred for use in the practice of the present invention is a copolymer of acrylic acid cross linked with approximately 0.75% to approximately 1.5% polyallyl sucrose that is sold under the trade designation Carbopol 934, 974 by Noveon of Cleveland, Ohio, USA.

Hydrophobic organic materials are also useful as adhesion enhancing agents in the practice of the present invention include hydrophobic materials such as waxes such as bees wax, plastigel, (a blend of mineral oil and polyethylene), petrolatum, white petrolatum, versagel (blend of liquid paraffin, butene/ethylene/styrene hydrogenated copolymer) acrylate and vinyl acetate polymers and copolymers, polyethylene waxes, silicone polymers such as dimethicone, silicone elastomers, organosiloxane resins, silicone gums and polyvinyl pyrrolidone/vinyl acetate copolymers.

The adhesion enhancing agent is present in various embodiments at from about 0.003% to about 10%, preferably from about 0.05% to about 1% by weight.

Carrier

In addition to the ingredients described above, the whitening composition also comprises a carrier, especially those whitening compositions that are in liquid solution. Some suitable carriers for use in the preparation of the whitening compositions of the present invention include volatile solvents such ethanol, ethyl acetate, and propyl acetate and non-volatile, water soluble solvents such as triacetin. Generally the solvent in which the whitening particle is dispersed is present in the composition at a concentration of about 0.1% to about 90% by weight and preferably about 40% to about 70% by weight.

In various embodiments, the carrier may also include an anti-adhesion agent to prevent adhesion of the whitening particulates to non-tooth surfaces (lips, tongue, etc.). Adhesion of the whitening particulates to the tongue, for example, may cause an unnatural look and detract from the natural white appearance imparted by the composition. The anti-adhesion agent may be added to the carrier to prevent such adhesion. An exemplary anti-adhesion agent is mineral oil. The mineral oil is present in an amount sufficient to prevent adhesion of the whitening agent to the non-tooth surfaces and does not substantially interfere with the ability of the whitening agent to impart a natural white appearance for a desired period of time. The agent may be present in the oral care composition at from about 0.1% to about 20% by weight.

Whitening Particulates

In various embodiments, the whitening composition comprises a whitening particulate. As used herein, the term “whitening particulate” can encompass a plurality of whiteness-imparting particulates or particles within the whitening composition. It should be noted that the plurality of particulates may comprise one or more different species of whitening particulate materials that are independently selected from one another. In various embodiments, the whitening particulate comprises hydroxyapatite and another whitening particulate. The selection of multiple whitening particles may be useful for modifying the overall color shade of the whitening composition. In various embodiments, the whitening particulates can be any white colored or white pigmented particles such as, for example, white mineral particles, white metal oxide particles, or a white polymer particles. As used herein, “white” is considered a color, and a “white” color can be any color commonly perceived as white, for example colors set forth in the Vita Shade Guide scale of whiteness, or colors that are perceived as whiter than those displayed in the Vita Shade Guide.

In various embodiments of the present invention, the average diameter of such particles can be from about 0.5 μm (micrometers) to about 500 μm, from about 10 μm to about 100 μm, or from about 20 μm to about 50 μm.

In some embodiments, white mineral particles can comprise a non-toxic mineral or salt that can impart a white color. In various embodiments, the whitening particulates can comprise a calcium phosphate. In various configurations, the calcium phosphate can have a structure selected from tetracalcium phosphate, amorphous calcium phosphate, alpha-tricalcium phosphate, beta-tricalcium phosphate and hydroxyapatite (Ca₅(OH)(PO₄)₃). The calcium phosphate in various embodiments, can be a substantially aqueous insoluble calcium phosphate and non-crystalline, poorly crystalline or crystalline form such as, for example, crystalline hydroxyapatite.

Hydroxyapatite has a similar physical structure as tooth enamel, and thus has a strong affinity to the tooth enamel surfaces, resulting in the hydroxyapatite particulates imparting a “natural” white appearance to the enamel surface, where they are used. The preparation of hydroxyapatite is well known to the art as disclosed in U.S. Pat. No. 4,274,879, to Irvine, issued Jun. 23, 1981; U.S. Pat. No. 4,330,514, to Nagai et al., issued May 18, 1982; U.S. Pat. No. 4,324,772, to Conn et al., issued Apr. 13, 1982; U.S. Pat. No. 4,408,300, to Shima, issued Oct. 4, 1983; U.S. Pat. No. 4,097,935, to Jarcho, issued Jul. 4, 1978; and U.S. Pat. No. 4,207,300, to Kestner et al., issued Jun. 10, 1980. The hydroxyapatite whitening agent is present in the whitening composition of the present invention at a concentration of about 0.5 to about 60% by weight and preferably about 15% to about 40% by weight.

Suitable hydroxyapatite whitening agents may be purchased from the Ikeda Corporation, Yokohama, Japan. In some configurations, hydroxyapatite particles can comprise aggregates of smaller hydroxyapatite particles. In non-limiting examples, such aggregates can have a mean diameter of from about 100 nm to about 1000 nm, and comprise hydroxyapatite particles having a mean diameter of from about 0.1 nm to about 10 nm.

The whitening particulates can be, in some embodiments, a metal oxide. The metal oxide can comprise any metal oxide that provides a white color, such as, for example, titanium oxide, aluminum oxide, tin oxide, calcium oxide, magnesium oxide, barium oxide, or a combination thereof.

The whitening particulates can be, in some embodiments, polymeric white-colored particles such as disclosed in U.S. Pat. No. 6,669,930, to Hoic et al, issued Dec. 30, 2003. Polymeric white-colored particles can comprise, in non-limiting example, polyethylene (PE), polypropylene, ethylene/propylene copolymer, polytetrafluoroethylene (PTFE) or polyhexafluoropropene. In a non-limiting example, the polymeric white-colored particles can be polyethylene PE220, polypropylene, or PTFE as supplied by PreSperse, Inc., Somerset N.J. In a non-limiting example, the polymeric whitening particulates can comprise a polymer having a weight average molecular weight, a number average molecular weight, a Z-average molecular weight or a viscosity average molecular weight ranging from about 100 to about 10,000,000; from about 200 to about 5,000,000; from about 500 to about 1,000,000; from about 1,000 to about 500,000; from about 10,000 to about 100,000, or from about 20,000 to about 50,000.

In some embodiments, the whitening particulates can comprise pearlescent particles. In some configurations, the pearlescent particles can provide a white pearlescent appearance to a composition herein. “Pearlescence” and “pearlescent,” as used herein, refers to an optical property of a material in which the material can have a pearl-like, lustrous appearance. In some configurations, a pearlescent material can provide an appearance of depth. In some aspects, a pearlescent material can further provide an appearance of shine. Without being limited by theory, pearlescent particles are believed to partially reflect and partially refract incident light. The extent of partial refraction or reflection of incident light by a pearlescent material can depend on the angle of light incidence and/or the angle of viewing. Pearlescent particles used in the compositions described herein can provide aesthetic or cosmetic effects such as, for example, sparkle or luster.

Pearlescent particles can comprise a single mineral or chemical species, such as, for example a silicate such as mica, or bismuth oxychloride. By “mica” is meant any one of a group of hydrous aluminum silicate minerals with platy morphology and perfect basal (micaceous) cleavage. Mica can be, for example, sheet mica, scrap mica or flake mica, as exemplified by muscovite, biotite or phlogopite type micas. In some embodiments, the pearlescent particles can comprise a complex comprising more than one mineral or chemical species, such as, for example, mica coated with a metal oxide such as titanium oxide. Pearlescent particles can also be of biological origin, for example fish scale or mother-of-pearl. Certain pearlescent particles of biological origin can comprise calcium carbonate, such as, for example, pearl, mollusk shell such as mother-of-pearl obtained from oyster shell, or nacre.

In some embodiments, white pearlescent particles can be, for example, those described as Timiron® pigments, Biron® powders, Biron® dispersions or Nailsyn® dispersions (all registered trademarks of EM Industries, Inc. Hawthorne, N.Y., division of E. Merck). For example, mica titanium particles can be pearlescent particles such as Timiron® particles. White pearlescent mica titanium particles can be, for example “Silverwhite” Timiron® particles such as Timiron® Starluster MP-115, Timiron® Supersheen MP-1001, Timiron® Sparkle MP-47, Timiron® Supersilk MP-1005, Timiron® Pearl Flake MP-10, Timiron® Pearl Sheen MP-30, Timiron® Super Silver Fine, Timiron® Gleamer Flake MP-111, Timiron® Ultraluster MP-45, Timiron® Transwhite MP-18, Timiron® Diamond Cluster MP-149, Timiron® Super Silver, Timiron® Stardust MP-80, Timiron® Arctic Silver or Timiron® Snowflake MP-99.

A tooth-coating fluid of the present invention can comprise whitening particulates from about 0.01% (w/w) up to about 60% (w/w), from about 0.5% (w/w) up to about 30% (w/w), or from about 15% (w/w) up to about 25% (w/w). In some configurations, the whitening particulates (including pearlescent particles) can have an average size of from about 0.01 μm (microns) to about 500 μm, from about 0.5 μm to about 100 μm, or from about 2 μm to about 20 μm in diameter or longest dimension. Such particles as referenced herein are intended to include primary particles as well as aggregates of particles.

The plurality of whitening particulates can be independently comprised of a material selected from the group consisting of calcium phosphate compounds, titanium oxide, aluminum oxide, tin oxide, calcium oxide, magnesium oxide, polyethylene, polypropylene, ethylene/propylene copolymer, polytetrafluoroethylene, polyhexafluoropropene and combinations thereof. In one embodiment, the plurality of whitening particulates can comprise a calcium phosphate, such as a hydroxyapatite, in particular a crystalline hydroxyapatite.

Additional Ingredients

In some embodiments, a sweetener is employed in the tooth whitening composition of the present invention. Sweeteners among those useful herein include orally acceptable natural or artificial, nutritive or non-nutritive sweeteners. Such sweeteners include dextrose, polydextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof, sucralose, dipeptide-based intense sweeteners, cyclamates, dihydrochalcones, and mixtures thereof. One or more sweeteners are optionally present in a total amount depending strongly on the particular sweetener(s) selected, but typically at levels of from about 0.01% to about 5% by weight, and preferably about 0.5% to about 2% by weight. Sodium saccharin is preferred for certain embodiments.

The tooth whitening compositions of the present invention optionally comprise a flavoring agent in various embodiments. Flavorants among those useful herein include any material or mixture of materials operable to enhance the taste of the composition. Any orally acceptable natural or synthetic flavorant can be used, such as flavoring oils, flavoring aldehydes, esters, alcohols, similar materials, and combinations thereof. Flavorants include vanillin, sage, marjoram, parsley oil, spearmint oil, cinnamon oil, oil of wintergreen (methyl salicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants, and mixtures thereof. Also encompassed within flavorants herein are ingredients that provide fragrance and/or other sensory effect in the mouth, including cooling or warming effects. Such ingredients include menthol, menthyl acetate, menthyl lactate, camphor, eucalyptus oil, eucalyptol, anethole, eugenol, cassia, oxanone, α-irisone, propenyl guaiethol, thymol, linalool, benzaldehyde, cinnamaldehyde, N-ethyl-p-menthan-3-carboxamine, N,2,3-trimethyl-2-isopropylbutanamide, 3-1-menthoxypropane-1,2-diol, cinnamaldehyde glycerol acetal (CGA), methone glycerol acetal (MGA), and mixtures thereof. One or more flavorants are optionally present in a total amount of about 0.03% to about 5% by weight, optionally in various embodiments from about 0.2 to about 1% by weight.

Optional Ingredients

In various embodiments, an active ingredient is included in the whitening compositions of the present invention. In some embodiments, the optional active ingredient is a “systemic active” which is operable to treat or prevent a disorder which, in whole or in part, is not a disorder of the oral cavity. In various embodiments, the active is an “oral care active” operable to treat or prevent a disorder or provide a cosmetic benefit within the oral cavity (e.g., to the teeth, gingiva or other hard or soft tissue of the oral cavity). Oral care actives among those useful herein include whitening agents, anticaries agents, tartar control agents, periodontal actives, abrasives, breath freshening agents, malodour control agents, tooth desensitizers, salivary stimulants, antibacterial agents, and combinations thereof. It is understood that while general attributes of each of the above categories of actives may differ, there may some common attributes and any given material may serve multiple purposes within two or more of such categories of actives.

Compositions of the present invention may also be used for the treatment or prevention of systemic disorders, such as the improvement of overall systemic health characterized by a reduction in risk of development of systemic diseases, such as cardiovascular disease, stroke, diabetes, severe respiratory infection, premature and low birth weight infants (including associated postpartum dysfunction in neurologic/developmental function), and associated increased risk of mortality. Such methods include those disclosed in U.S. Patent Publication 2003/0206874, to Doyle et al., published Nov. 6, 2003.

The compositions of the present invention optionally comprise an antimicrobial (e.g., antibacterial) agent. One or more of such agents can be present. Suitable examples include without limitation copper (II) compounds such as copper (II) chloride, fluoride, sulfate and hydroxide, zinc ion sources such as zinc acetate, zinc citrate, zinc gluconate, zinc glycinate, zinc oxide, zinc sulfate and sodium zinc citrate, phthalic acid and salts thereof such as magnesium monopotassium phthalate, hexetidine, octenidine, sanguinarine, benzalkonium chloride, domiphen bromide, alkylpyridinium chlorides such as cetylpyridinium chloride (CPC) (including combinations of CPC with zinc and/or enzymes), tetradecylpyridinium chloride and N-tetradecyl-4-ethylpyridinium chloride, iodine, sulfonamides, bisbiguanides such as alexidine, chlorhexidine and chlorhexidine digluconate, piperidino derivatives such as delmopinol and octapinol, magnolia extract, grapeseed extract, menthol, geraniol, citral, eucalyptol, antibiotics such as augmentin, amoxicillin, tetracycline, doxycycline, minocycline, metronidazole, neomycin, kanamycin and clindamycin, and the like.

Other suitable antibacterial agents include non-ionic and anionic antibacterial agents known to one of skill in the art. Example non-ionic antibacterial agents include the substantially water insoluble, noncationic antibacterial agents such as alkylphenoxy phenols; cycloalkyl-phenoxyphenols; 9,10-dihydrophenanthrenol; alkylphenols; cycloalkyl-phenols; phenolic compounds; halogenated carbanilides; halogenated salicylanilides; benzoic esters; halogenated diphenyl ethers, and mixtures thereof. A particularly suitable non-ionic antibacterial agent is a diphenyl ether such as 2,4,4′-trichloro-2′-hydroxydiphenyl ether (Triclosan) and 2,2′-dihydroxy-5,5′-dibromodiphenyl ether. A further illustrative list of useful antibacterial agents is provided in U.S. Pat. No. 5,776,435 to Gaffar et al., issued Jul. 7, 1998 incorporated herein by reference. One or more antimicrobial agents are optionally present in an antimicrobial effective total amount, typically about 0.05% to about 10%, for example about 0.1% to about 3% by weight, of the composition.

The compositions of the present invention optionally comprise an antioxidant. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

The compositions of the present invention optionally comprise an orally acceptable zinc ion source useful, for example, as an antimicrobial, anticalculus or breath-freshening agent. One or more such sources can be present. Suitable zinc ion sources include without limitation zinc acetate, zinc citrate, zinc gluconate, zinc glycinate, zinc oxide, zinc sulfate, sodium zinc citrate and the like. One or more zinc ion sources are optionally and illustratively present in a total amount of about 0.05% to about 3%, for example about 0.1% to about 1%, by weight of the composition.

The compositions of the present invention optionally comprise an antiplaque (e.g., plaque disrupting) agent. One or more such agents can be present in an antiplaque effective total amount. Suitable antiplaque agents include without limitation stannous, copper, magnesium and strontium salts, dimethicone copolyols such as cetyl dimethicone copolyol, papain, glucoamylase, glucose oxidase, urea, calcium lactate, calcium glycerophosphate, strontium polyacrylates and chelating agents such as citric and tartaric acids and alkali metal salts thereof.

The compositions of the present invention optionally comprise an anti-inflammatory agent. One or more such agents can be present in an anti-inflammatory effective total amount. Suitable anti-inflammatory agents include without limitation steroidal agents such as flucinolone and hydrocortisone, and nonsteroidal agents (NSAIDs) such as ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone and phenylbutazone. One or more anti-inflammatory agents are optionally present in the composition in an anti-inflammatory effective amount.

The compositions of the present invention optionally comprise an H₂ histamine receptor antagonist. H₂ antagonists useful herein include cimetidine, etintidine, ranitidine, ICIA-5165, tiotidine, ORF-17578, lupititidine, donetidine, famotidine, roxatidine, pifatidine, lamtidine, BL-6548, BMY-25271, zaltidine, nizatidine, mifentidine, BMY-52368, SKF-94482, BL-6341A, ICI-162846, ramixotidine, Wy-45727, SR-58042, BMY-25405, loxtidine, DA-4634, bisfentidine, sufotidine, ebrotidine, HE-30-256, D-16637, FRG-8813, FRG-8701, impromidine, L-643728, HB-408.4, and mixtures thereof.

The compositions of the present invention optionally comprise a desensitizing, or tooth sensitivity protecting, agent. One or more such agents can be present. Suitable desensitizing agents include without limitation potassium salts such as potassium citrate, potassium tartrate, potassium chloride, potassium sulfate and potassium nitrate. Another suitable desensitizing agent is sodium nitrate. Alternatively or in addition a local or systemic analgesic such as aspirin, codeine, acetaminophen, sodium salicylate or triethanolamine salicylate can be used. One or more desensitizing agents and/or analgesics are optionally present in a desensitizing and/or analgesic effective amount, typically about 0.05% to about 5%, for example about 0.1% to about 3% by weight, of the composition.

The compositions of the present invention optionally comprise a nutrient. Suitable nutrients include vitamins, minerals, amino acids, and mixtures thereof. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophane, L-lysine, methionine, threonine, levocarnitine and L-carnitine), lipotropics (such as choline, inositol, betaine, and linoleic acid), fish oil (including components thereof such as omega-3 (N-3) polyunsaturated fatty acids, eicosapentaenoic acid and docosahexaenoic acid), coenzyme Q10, and mixtures thereof.

The compositions of the present invention optionally comprise proteins. Suitable proteins include milk proteins and enzymes such as peroxide-producing enzymes, amylase, plaque-disrupting agents such as papain, glucoamylase, and glucose oxidase.

The compositions of the present invention optionally comprise additional ingredients, such as, plaque buffers such as urea, calcium lactate, calcium glycerophosphate and strontium polyacrylates; plant extracts; opacifying agents, and additional pigments and/or coloring agents.

The whitening composition of the present invention may be applied to tooth enamel by any suitable means, such as for example, in extruded form as a strip or as a liquid such as a paint-on liquid.

Strip

The whitening composition of the present invention may be in the form of a layer of a strip prepared using a conventional solvent casting process. Strips among those useful herein comprise polymers, natural and synthetic woven materials, non-woven material, foil, paper, rubber and combinations thereof. Preferably the strip of material is substantially water insoluble. Suitable polymers include polyethylene, ethylvinylacetate, polyesters, ethylvinyl alcohol, fluoroplastics, and combinations thereof. In various embodiments, the strip of material is generally less than about 1 mm (millimeter) thick, optionally less than about 0.05 mm thick, optionally about 0.001 to about 0.03 mm thick. The shape of the strip is any shape and size that covers the desired oral surface. In one embodiment, the length of the strip material is from about 2 cm (centimeter) to about 12 cm, in another embodiment from about 4 cm to about 9 cm. The width of the strip material will also depend on the oral surface area to be covered. The width of the strip is generally from about 0.5 cm to about 4 cm, in one embodiment from about 1 cm to about 2 cm. The strip material may comprise shallow pockets, optionally filled by a composition of this invention. Strips among those useful herein are disclosed in U.S. Pat. No. 6,514,484, to Rajaiah et al. issued Feb. 4, 2003.

For example, in some embodiments, a strip is prepared by solvent casting an adhesive polymer or resin such as colophonium and/or polyvinylpyrrolidone which is dissolved in a sufficient amount of a compatible solvent such as ethanol. After a solution has been formed, the addition of whitening particulates in powder form (e.g., hydroxyapatite) follows and any other ingredients such as sweeteners or flavors. The solution is coated onto a suitable casting carrier material from which the formed strip can be easily released from without damage. The carrier material must have a surface tension which allows the solution to spread evenly across the intended carrier width without soaking in to form a destructive bond between the two substrates. Examples of suitable carrier materials include glass, stainless steel, PTFE (Teflon), polyethylene impregnated kraft paper. The strip may be dried to a solid usable form in a high temperature air bath using a drying oven, drying tunnel, vacuum drier, or any other suitable drying equipment at a temperature. Thereafter the strip formed on the carrier is peeled off the carrier surface and cut into pieces of suitable size and shape for consumer use and packed into a suitable container.

To use the whitening strip for the present invention, the strip when applied to the teeth surface will adhere to the teeth in an appropriate manner to provide a natural appearance as the whitening particulates present in the strip matrix mask the stained or darker color of the tooth surfaces. In this regard, the whitening strip is formed to have a width dimension suitable to cover one or more teeth in a row (upper or lower). Therefore, the whitening strip may be applied to one or more of the upper set of teeth, or to one or more of the lower set of teeth either separately or simultaneously. The length dimension of the whitening strip is determined by the amount of coverage desired. In this regard, the number of teeth which it is desired to whiten will determine the dimensions for the whitening strip. For instance, it may be desired to only whiten the front teeth, which are most easily seen by others. Accordingly, the length of whitening strip can be reduced in this case, as compared to the case where it is desired to whiten all of the teeth. The duration of application of whitening strip to the teeth will depend upon the type and concentration of the whitening particulate, as well as the type and intensity of existing discoloration. The strip incorporating the whitening particulates will preferably resist removal by salivation, by beverages and food, or mastication for at least 12 to 18 hours and generally 3 to 5 days.

Paint-On Tooth Whitener

In one preferred embodiment, the whitening composition is applied using a “paint-on” technique. A small application device, such as a brush or spatula is coated with a composition of this invention and the composition is then placed on a tooth surface. Preferably, the composition is spread evenly on such surfaces, in sufficient quantity to deliver whitening particulates to the stained surfaces.

The paint-on whitening composition of the present invention is prepared in the form of a flowable viscous liquid suspension containing the whitening particulate and is applied as such to the subject's teeth, by manual application, such as by painting the teeth with a soft applicator brush in the same manner as application of nail polish to a finger nail and without the intervention of a dentist or technological operations. Application by the user and evaporation or dissolution of the solvent leaves an adherent natural appearing white coating on the teeth which provides almost instantaneous whitening of the tooth enamel.

In various embodiments, the layer of tooth whitening composition applied to tooth enamel contains no ingredients imparting thereto an unacceptable taste or texture, rendering it unpleasant to the user while adhering strongly to tooth enamel. The composition is sufficiently adherent to tooth enamel to remain on the teeth for the applied whitening composition enabling the applied coating to resist the forces commonly applied by the lips and tongue as well a those forces encountered during normal mastication, as upon the evaporation or dissolution of the solvent in mouth after application a hard coating of whitening particulate forms in about 1 minute which coating is bonded securely to the tooth enamel to which it is applied.

### METHODS

A method of imparting whiteness to a tooth in a mammal is provided in one embodiment of the present invention, where the method comprises applying a composition to the tooth. The composition comprises a silicone resin, a silicone pressure sensitive adhesive, and a whitening particulate, such as those previously described. In certain embodiments, the siloxane pressure sensitive adhesive comprises a copolymer of polyorganosiloxane and silicone resin. Preferred whitening particulates are one or more materials selected from the group consisting of calcium phosphate, titanium oxide, aluminum oxide, tin oxide, calcium oxide, magnesium oxide, polyethylene, polypropylene, a copolymer of ethylene/propylene, polytetrafluoroethylene, polyhexafluoropropene and combinations thereof. Other preferred whitening particulates are selected from the group consisting of: tetracalcium phosphate, amorphous calcium phosphate, alpha-tricalcium phosphate, beta-tricalcium phosphate, hydroxyapatite and mixtures thereof. The whitening particulate preferably has an average diameter of from at least about 0.5 micrometers up to about 500 micrometers.

In an embodiment, the applying is achieved by contacting a film comprising the tooth whitening composition with a surface of a the tooth. In an alternate embodiment, the applying is achieved by contacting a liquid form of the tooth whitening composition with a surface of the tooth.

The whitening composition of the present invention is prepared by adding the ingredients of the composition in a suitable vessel such as a stainless steel tank provided with a mixer in one embodiment. In the preparation for the whitening composition, the ingredients are advantageously added to the mixer in the following order: solvent, silicone adhesive materials, silicone resin, hydroxyapatite and any desired flavoring or sweetener. The ingredients are then mixed to form a homogeneous dispersion. Optionally, the silicone adhesive and hydroxyapatite may be combined to form a first mixture and the silicone resin and solvent combined to form a second mixture. The first and second mixtures are then combined and the desired flavoring, sweetener, etc. are added to the mixture. When mixed and ready for extrusion or application directly as a paint-on whitener, the composition is a white colored opaque viscous liquid composition.

The present invention is illustrated by the following examples but is not to be limited thereby.

### EXAMPLES

### Example 1

A liquid paint on instant tooth whitening composition is prepared according to Table 1. The composition is painted onto the dental enamel of a subject. The composition dries in 1.5 minutes. The composition does not adhere to non-enamel oral surfaces.

Table 1

TABLE 1

| Ingredient | Weight Percentage |
|---|---|
| BIO-PSA Silicone Adhesive | 20 |
| Medium Tack Silicone Adhesive in Amine | 20 |
| Trimethylsiloxysilicate | 14 |
| Mineral Oil | 10 |
| Propyl Acetate | 15 |
| Hydroxyapatite | 20 |
| Flavor | 0.6 |
| Saccharin | 0.4 |
| Total | 100% |

### Example 2

A liquid paint on instant tooth whitening composition is prepared according to the following Table 2. The composition is painted onto the dental enamel of a subject. The composition dries in 1.5 minutes and imparts a natural white appearance to the teeth for 5 days.

TABLE 2

| Ingredient | Weight Percentage |
|---|---|
| BIO-PSA Silicone Adhesive | 20 |
| Silicone Adhesive in Dimethicone | 20 |
| Trimethylsiloxysilicate | 10 |
| Hydroxyapatite | 18 |
| Flavor | 0.6 |
| Ethylacetate | 31.05 |
| Saccharin | 0.3 |
| Fumed Silica | 0.05 |
| Total | 100% |

What is claimed is:

1. A tooth whitening composition for imparting a natural white appearance to dental enamel, comprising:
a. a whitening particulate comprising hydroxyapatite; and
b. a delivery system comprising a silicone resin and a silicone adhesive.

2. A tooth whitening composition according to claim 1, wherein said whitening particulate is selected from the group consisting of: calcium phosphate, titanium oxide, aluminum oxide, tin oxide, calcium oxide, magnesium oxide, polyethylene, polypropylene, a copolymer of ethylene/propylene, polytetrafluorethylene, polyhexafluoropropene, and combinations thereof.

3. A tooth whitening composition according to claim 2, wherein said whitening particulate is selected from the group consisting of: hydroxyapatite, tetracalcium phosphate, amorphous calcium phosphate, alpha-tricalcium phosphate, beta-tricalcium phosphate, and mixtures thereof.

4. A tooth whitening composition according to claim 1, wherein said whitening particulate has an average diameter of from at least about 0.5 micrometers up to about 500 micrometers.

5. A tooth whitening composition according to claim 1, wherein said silicone resin comprises trimethylsiloxysilicate.

6. A tooth whitening composition according to claim 1, wherein said silicone adhesive is a copolymer of a silicone resin and an organosiloxane.

7. A tooth whitening composition according to claim 1, wherein said delivery system comprises a siloxane pressure sensitive adhesive.

8. A tooth whitening composition according to claim 1, wherein said delivery system comprises a silicone adhesive having a terminal group selected from the group consisting of amine terminal groups and hydroxyl terminal groups.

9. A tooth whitening composition according to claim 1, wherein said whitening particulate comprises hydroxyapatite, said silicone resin comprises trimethylsiloxysilicate, and said silicone adhesive comprises a pressure sensitive adhesive and an additional silicone adhesive having a terminal group selected from the group consisting of amine terminal groups and hydroxyl terminal groups.

10. A tooth whitening composition according to claim 1, comprising at least two silicone adhesives each having a different adhesive tack level from one another.

11. A tooth whitening composition according to claim 1, wherein said delivery system further comprises an additional polymer selected from the group consisting of natural resins, modified natural resins, and pressure sensitive adhesives.

12. A tooth whitening composition according to claim 1, wherein said composition comprises from about 0.1% to about 99% by weight of said delivery system and from about 0.5% to about 60% by weight of said whitening particulate.

13. A tooth whitening composition according to claim 1, wherein said delivery system further comprises an ingredient selected from the group consisting of: solvents, thickeners, adhesion enhancing agents, and combinations thereof.

14. A dental strip comprising a layer comprising the composition according to claim 1, wherein said layer is operable to adhere to a tooth surface.

15. A liquid tooth whitening composition comprising the composition according to claim 1, wherein said liquid tooth whitening composition is operable to adhere to a surface of a tooth.

16. A method for imparting whiteness to a tooth in a mammal, comprising applying to the tooth a composition, comprising:
a. a whitening particulate comprising hydroxyapatite; and
b. a delivery system comprising a silicone resin and a silicone adhesive.

17. A method according to claim 16, wherein said applying is achieved by contacting a liquid form of said tooth whitening composition with a surface of the tooth.

18. A method according to claim 16, wherein said applying is achieved by contacting a film comprising said tooth whitening composition with a surface of the tooth.

19. A method according to claim 16, wherein said silicone resin comprises trimethylsiloxysilicate.

20. A method according to claim 16, wherein said delivery system comprises at least two silicone adhesives each having a different adhesive tack level from one another.

21. A method according to claim 16, wherein said whitening particulate is comprised of one or more materials selected from the group consisting of calcium phosphate, titanium oxide, aluminum oxide, tin oxide, calcium oxide, magnesium oxide, polyethylene, polypropylene, a copolymer of ethylene/propylene, polytetrafluorethylene, polyhexafluoropropene, and combinations thereof.

22. A method according to claim 16, wherein said whitening particulate is selected from the group consisting of: hydroxyapatite, tetracalcium phosphate, amorphous calcium phosphate, alpha-tricalcium phosphate, beta-tricalcium phosphate, and mixtures thereof.

23. A tooth whitening composition, comprising:
a. a whitening particulate comprising hydroxyapatite; and
b. a delivery system comprising trimethylsiloxysilicate and at least one silicone adhesive at a ratio that provides a strong and flexible adhesive layer, and wherein after application to a tooth surface, the composition dries to form a durable layer over said tooth.
